(19) United States
(12) Patent Application Publication
(10) Pub. No.: US 2005/0209231 A1
(43) Pub. Date: Sep. 22, 2005

(54) Compositions and methods for inducing cardiomyogenesis

(76) Inventors: Xu Wu, San Diego, CA (US); Sheng Ding, San Diego, CA (US); Peter G. Schultz, La Jolla, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/035,708

(22) Filed: Jan. 14, 2005

Related U.S. Application Data
(60) Provisional application No. 60/537,144, filed on Jan. 16, 2004.

(51) Int. Cl. A61K 31/5377; A61K 31/506; C07D 413/02; C07D 43/02

(52) U.S. Cl. 514/235.5; 514/275; 544/122; 544/294; 544/324

(57) ABSTRACT

The present invention provides compositions and methods for inducing cardiomyogenesis in mammalian cells, particularly embryonic stem cells, in vitro and in vivo.

### CROSS-REFERENCE TO RELATED APPLICATIONS

The present application claims priority to U.S. Provisional Patent Application No. 60/537,144, filed Jan. 16, 2004, the teachings of which are hereby incorporated by reference in their entirety.

### BACKGROUND OF THE INVENTION

Heart disease is a major problem throughout the world, encompassing many different illnesses and conditions. Cardiomyopathy, for example, is a disease of the heart muscle wherein the heart loses its ability to pump blood and, in some instances, heart rhythm is disturbed, leading to irregular heartbeats, or arrhythmias. Cardiomyopathy affects tens of thousands of Americans of all ages and is a leading reason for heart transplantation. The condition tends to be progressive and sometimes worsens fairly quickly.

Understanding the development and function of cardiac muscle would be facilitated by the use of stem cells. Stem cells are multipotent cells with the ability to self-renew and differentiate into specialized cells in response to appropriate signals. See, e.g., Spradling et al., Nature, 414:98-104 (2001). Most tissues have endogenous stem/progenitor cells which upon injury to the organ, can proliferate and differentiate at the damaged site. The adult heart, however, is composed mainly of post-mitotic and terminally differentiated cells. Although a subpopulation of myocardial cells with cardiac stem cell character was identified recently, their limited availability hinders therapeutic applications. See, e.g., Beltrami et al., Cell, 114:763-776 (2003). Stem cells derived from other tissues, such as bone marrow, have been shown to be capable of repairing heart damage in animal models' but inefficient differentiation and possible fusion with somatic cells limit their use in cardiac repair. See, e.g., Ferrari et al., Science, 279:1528-30 (1998).

Pluripotent embryonic stem (ES) cells represent a possible unlimited source of functional cardiomyocytes. Such cardiomyocytes would likely facilitate the therapeutic application of ES cells in heart disease, as well as provide important tools for probing the molecular mechanism of cardiomyocyte differentiation and heart development. To date, however, the in vitro differentiation of ES cells into cardiomyocytes involves a poorly defined, inefficient and relatively non-selective process. See, e.g., Boheler et al., Circ. Res., 91:189-201 (2002).

Thus, the art recognizes a need for compositions and methods for inducing and directing the differentiation of ES cells into cardiomyocytes. There is a particular need for small molecules that can induce in vivo and in vitro differentiation of ES cells into cells of a myocardial lineage. This invention satisfies these and other needs.

### SUMMARY OF THE INVENTION

The present invention provides novel compositions and methods for inducing and directing the differentiation of ES cells into cells of a myocardiac lineage.

One embodiment of the invention provides compounds of Formula I having the following structure: 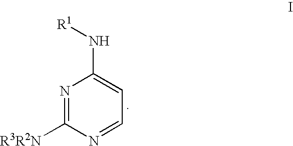
In Formula I, R¹ is a functional group including, but not limited to, hydrogen, C₁₋₄alkyl, C₃₋₈cycloalkyl, and C₀₋₂alkylaryl, substituted with 0-2 R^{1a} groups that are independently selected and are functional groups, including, but not limited to, halogen, C₁₋₄alkyl, C₁₋₄alkoxy, —OH, —N(R^{1b}, R^{1b}), —SO₂N(R^{1b}, R^{1b}), —C(O)N(R^{1b}, R^{1b}), heterocycloalkyl and —O-aryl, or when R^{1a} groups are on adjacent ring atoms, they are optionally taken together to form a functional group including, but not limited to, —O—(CH₂)₁₋₂—O—, —O—C(CH₃)₂CH₂— and —(CH₂)₃₋₄—, or R¹ is optionally taken together with the nitrogen to which it is attached to form a heterocycle, optionally substituted with C₁₋₄alkyl, C₃₋₈cycloalkyl, C₁₋₄alkylhydroxy and C₀₋₂alkylaryl; each R^{1b} group is independently selected and is a functional group including, but not limited to, hydrogen and C₁₋₄alkyl. In Formula I, R² is a functional group including, but not limited to, C₁₋₄alkyl, C₃₋₈cycloalkyl and CO₂alkylaryl, substituted with 0-2 R groups. Group R^{2a} is independently selected and is a functional group including, but not limited to, halogen, C₁₋₄alkyl, C₁₋₄alkoxy, —N(R^{2b}, R^{2b}), —SO₂N(R^{2b}, R^{2b}), —C(O)N(R^{2b}, R^{2b}) and —O-aryl, or when R^{2a} groups are on adjacent ring atoms, they are optionally taken together to form a functional group including, but not limited to, —O—(CH₂)₁₋₂—O—, —O—C(CH₃)₂CH₂— and —(CH₂)₃₋₄—; and each R^{2b} group is independently selected and is a functional group including, but not limited to, hydrogen and C₁₋₄alkyl. R³, in Formula I, is typically hydrogen, or R³ is optionally taken together with R² and the nitrogen to which both are attached to form a heterocycle, optionally substituted with, for example, C₁₋₄alkyl or C₀₋₂alkylaryl.

The compounds of the present invention include all pharmaceutically acceptable salts, isomers, solvates, hydrates and prodrugs thereof.

In another embodiment, the present invention provides methods of inducing cardiomyogenesis. Mammalian cells are contacted with a compound of Formula I or II, whereupon the mammalian cell differentiates into a cell of a myocardiac lineage. The step of contacting can be in vivo or in vitro. In view of their ability to induce cardiomyogenesis, the compounds of Formula I or II are useful for treating cardiac muscle disorders, such as cardiomyopathy and arrhythmia, and for repairing heart muscle tissue damage resulting from a heart attack, for example.

Another embodiment of the present invention provides methods of treating cardiac muscle disorders by contacting a mammalian cell with a compound of Formula I, whereupon the mammalian cell differentiates into a cell of a myocardiac lineage. The mammalian cell may be further contacted with other compounds or proteins favorable to cardiomyogenesis. If the mammalian cell is contacted with a compound of Formula I or II in vitro, the differentiated cells are administered to an individual with a treatable disorder, thereby treating the disorder. In some embodiments, the mammalian cell is attached to a solid support (e.g., a three-dimensional matrix or a planar surface) or injected to the damaged sites of myocardium.

In some embodiments, the mammalian cell is contacted with a compound of Formula I or II in vivo. If the mammalian cell is contacted with a compound of Formula I or II in vivo, the step of contacting may be by oral, intravenous, subcutaneous, or intraperitoneal administration of the compound to the mammal.

In some embodiments, the differentiation of the mammalian cell into a cell of a myocardiac lineage is detected. In some embodiments, the differentiation of the mammalian cell into a cell of a myocardioblast is detected by detecting expression of a caridiomyogenesis marker gene, e.g., atrial natriuretic factor (“ANF”). In other embodiments, the differentiation of the mammalian cell into a cell of a myocardiac lineage is detected by detecting expression of a cardiac muscle cell-specific transcription factor (e.g., MEF2 or Nkx2.5 or the homeodomain transcription factor HOP). In other embodiments, the differentiation of the mammalian cell into a cell of a myocardiac lineage is detected by detecting expression of a cardiac muscle specific gene (e.g., myosin light chain 2V or eHAND). In still other embodiments, the differentiation of the mammalian cell into a cell of a myocardiac lineage is detected by detecting expression of a cardiac specific gene, such as GATA-4, or by the expression of a gene involved in cardiac muscle contractibility, such as the sarcomeric myosin heavy chain (MHC). In further embodiments, the differentiation may be detected by observing the beating of cardiac muscle using standard techniques well-known to those in the art.

In some embodiments, the mammalian cell is a stem cell (e.g., an embryonic stem cell or an embryonic carcinoma cell). In some embodiments, the stem cell is isolated from a mouse (e.g., a murine undifferentiated R1 embryonic stem cell or a murine carcinoma P19 cell) or from a primate (e.g., a human).

In some embodiments of the methods above, the compound administered to the mammalian cells is cardiogenol A, B, C or D, or a composition comprising one or more of cardiogenol A, B, C or D.

Other embodiments and advantages of the present invention will be apparent from the detailed description that follows.

### BRIEF DESCRIPTION OF THE DRAWINGS

FIG. 1. A high throughput assay for cardiomyogenesis using an ANF-promoter reporter assay. This figure shows data obtained using a stable P19 clone harboring an ANF promoter reporter plasmid expressing luciferase. The graph shows a 5- to 7-fold increase in luciferase signal from this P19 clone after several days under standard cardiomyogenesis differentiation conditions for P19 cells (EB formation and treatment with 1% DMSO (see Skerjank I S, Trends Cardiovasc Med, 9:139-143 (1999)).

FIG. 2. Immunostaining of cardiac muscle markers in ESCs (A to E) and P19CL6 cells (F) treated with 0.25 μM cardiogenol C: (A) and (F) Myosin Heavy Chain (green); (B) GATA-4 (red); (C) MEF2 (red); (D) Nkx2.5 (red); and (E) Myosin Heavy Chain (green) and MEF2 (red). Cell nuclei were stained with DAPI (blue). Cells were fixed with 4% paraformaldehyde (Sigma) for 20 min. Cell staining was performed in PBS (Gibco) with 0.3% Triton X-100 and 6% horse serum. Primary antibodies were used at the following dilutions: myosin heavy chain (MHC) mouse monoclonal antibody MF20 (Developmental Studies Hybridoma Bank, 1:200), rabbit polyclonal anti-GATA-4 antibody (Santa Cruz Biotech, 1:300), rabbit anti-MEF2 antibody (Santa Cruz Biotech, 1:100) and goat anti-Nkx2.5 antibody (Santa Cruz Biotech, 1:100). Secondary antibodies were Cy2-conjugated anti-mouse (1:300), or Cy3-conjugated anti-rabbit or anti-goat antibodies (Jackson ImmunoResearch, 1:500). Cell nuclei were stained with DAPI (Roche). Images were taken with a Nikon Eclipes TE2000 microscope with 200-fold magnification. Double or triple-labeled images were assembled in Metamorph.

FIG. 3. Immunostaining of ESCs without cardiogenol C treatment (control). (A). MHC (green) and Nuclei (Blue). (B). GATA-4 (Red) and Nuclei (Blue). Compare with FIGS. 2A and 2E, respectively.

FIG. 4. This figure shows a list of additional compounds of the invention that may be used for inducing cardiomyogenesis in mammalian cells.

### DETAILED DESCRIPTION OF THE INVENTION

I. Introduction

The present invention provides compounds, compositions and methods for differentiating mammalian cells into cells of a myocardiac lineage. More particularly, the present invention provides compounds of Formula I and II that are useful for differentiating mammalian cells into cells of a myocardiac lineage. In some embodiments, a composition comprising the compound of Formula I or II is provided. In other embodiments, methods of inducing cardiomyogenesis in mammalian cells are provided. Myogenesis can be induced in vivo or in vitro according to the methods of the present invention.

II. Definitions

Unless defined otherwise, all technical and scientific terms used herein generally have the same meaning as commonly understood by one of ordinary skill in the art to which this invention belongs. Generally, the nomenclature used herein and the laboratory procedures for organic and analytical chemistry are those well known and commonly employed in the art.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below as “heteroalkyl.” Alkyl groups which are limited to hydrocarbon groups are termed “homoalkyl”.

The term “alkylene” by itself or as part of another substituent means a divalent radical derived from an alkane, as exemplified by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—O—CH₂—CH₂—(CH₃)₂, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified by —CH₂—CH₂—S—CH₂CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include trifluoromethyl, 2,2,2-trifluoroethyl, 4-chlorobutyl, 3-bromopropyl, and the like.

The term “aryl” means, unless otherwise stated, a polyunsaturated, typically aromatic, hydrocarbon substituent which can be a single ring or multiple rings (up to three rings) which are fused together or linked covalently. The term “heteroaryl” refers to aryl groups (or rings) that contain from zero to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from: —OR′, ═O, ═NR′, ═N—OR′, —NR′R″, —SR′, -halogen, —SiR′R″ R′″, —OC(O)R′, —C(O)R′, —CO₂R′, —CONR′R″, —OC(O)NR′ R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —CN and —NO₂ in a number ranging from zero to (2m′+1), where m′ is the total number of carbon atoms in such radical. R′, R″ and R′″ each independently refer to hydrogen, unsubstituted (C₁-C₈)alkyl and heteroalkyl, unsubstituted aryl, aryl substituted with 1-3 halogens, unsubstituted alkyl, alkoxy or thioalkoxy groups, or aryl-(C₁-C₄)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. From the above discussion of substituents, one of skill in the art will understand that the term “alkyl” is meant to include groups such as haloalkyl (e.g., —CF₃ and —CH₂CF₃) and acyl (e.g., —C(O)CH₃, —C(O)CF₃, —C(O)CH₂OCH₃, and the like).

Similarly, substituents for the aryl and heteroaryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′ R″, —NR″C(O)R′, —NR″C(O)₂R′, —NR′—C(O)NR′R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₙ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted (C₁-C₆)alkyl.

The terms “halo” or “halogen” as used herein refer to Cl, Br, F or I substituents. The term “haloalkyl”, and the like, refer to an aliphatic carbon radicals having at least one hydrogen atom replaced by a Cl, Br, F or I atom, including mixtures of different halo atoms. Trihaloalkyl includes trifluoromethyl and the like as preferred radicals, for example.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

As used herein, the term “heteroatom” is meant to include oxygen (O), nitrogen (N) and sulfur (S).

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts,” Journal of Pharmaceutical Science, 66:1-19 (1977)). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

“Cardiomyogenesis,” as used herein, refers to the differentiation of progenitor or precursor cells into cardiac muscle cells (i.e., cardiomyocytes) and the growth of cardiac muscle tissue. Progenitor or precursor cells can be pluripotent stem cells such as, e.g., embryonic stem cells. Progenitor or precursor cells can be cells pre-committed to a myocardiac lineage (e.g., pre-cardiomyocyte cells) or cells that are not pre-committed (e.g., multipotent adult stem cells).

A “stem cell,” as used herein, refers to any self-renewing pluripotent cell or multipotent cell or progenitor cell or precursor cell that is capable of differentiating into multiple cell types. Stem cells suitable for use in the methods of the present invention include those that are capable of differentiating into cells of myocardiac lineage, e.g., cardiomyocytes. Suitable stem cells for use in the methods of the present invention include, for example, embryonic stem cells (“ESCs”) and embryonic carcinoma (“EC”) cells. Pluripotent embryonic stem cells are capable of differentiating into all types of tissue, including neuronal cells, muscle cells, blood cells, etc. See, e.g., Spradling et al. (2001).

“Differentiate” or “differentiation,” as used herein, refers to the process by which precursor or progenitor cells (i.e., stem cells) differentiate into specific cell types, e.g., cardiomyocytes. A differentiated cell can be identified by a number of features that are unique or distinctive with respect to that particular cell type. For example, differentiated cells may be identified by their patterns of gene expression and protein expression. Typically, cells of a myocardiac lineage express genes such as, for example, the sarcomeric myosin heavy chain, myosin light chain 2V, eHAND and ANF. See, e.g., Small et al., Cell, 110:725-735 (2002); Shin et al., Cell, 110:725-35 (2002). Also typically expressed by cells of a myocardiac lineage are cardiac muscle cell specific transcription factors such as MEF2, Nkx2.5 or the homeodomain transcription factor HOP. See, e.g., Edmondson et al., Development, 1251-1263 (1994); Lin et al., Science, 276:1404-1407 (1997). Additional transcription factors that are involved in cardiomyocyte differentiation include, e.g., GATA4 (see, e.g., Grepin et al., Development, 124:2387-95 (1997)). One skilled in the art will recognize that other cardiac muscle specific genes may be utilized to monitor and determine differentiation.

A “cardiomyocyte marker gene” is a gene which is expressed uniquely by developing cardiomyocytes or only rarely by other cell types, such that the marker gene is useful for the determination of whether a cell is a cardiomyocyte. An example of a cardiomyocyte marker gene is ANF, a polypeptide hormone that is synthesized primarily in cardiac myocytes and is a down-stream target of several cardiomyogenesis transcriptional factors.

A “solid support,” as used herein in connection with inducing cardiomyogenesis, refers to a three-dimensional matrix or a planar surface on which the stem cells can be cultured. The solid support can be derived from naturally occurring substances (i.e., protein based) or synthetic substances. For example, matrices based on naturally occurring substances may be composed of autologous bone fragments or commercially available bone substitutes as described in e.g., Clokie et al., J. Craniofac. Surg. 13(1): 111-21 (2002) and Isaksson, Swed. Dent. J. Suppl., 84:1-46 (1992). Suitable synthetic matrices are described in, e.g., U.S. Pat. Nos. 5,041,138, 5,512,474, and 6,425,222. For example, biodegradable artificial polymers, such as polyglycolic acid, polyorthoester, or polyanhydride can be used for the solid support. Calcium carbonate, aragonite, and porous ceramics (e.g., dense hydroxyapatite ceramic) are also suitable for use in the solid support. Polymers such as polypropylene, polyethylene glycol, and polystyrene can also be used in the solid support. Cells cultured and differentiated on a solid support that is a three-dimensional matrix typically grow on all of the surfaces of the matrix, e.g., internal and external. Cells cultured and differentiated on a solid support that is planar typically grow in a monolayer. The term “solid-support” is also used in the context of preparing the compounds of Formula I. In this context, “solid-support” refers to a polymeric support, such as a bead, that can be partially soluble in a suitable solvent or completely insoluble, and is used to bind, for example, a reactant or a reagent of the reaction. Suitable solid-supports include, but are not limited to, PAL resin, Wang resin, and polystyrene resin.

“Culturing,” as used herein, refers to maintaining cells under conditions in which they can proliferate, differentiate, and avoid senescence. For example, in the present invention, cultured embryonic stem cells proliferate and differentiate into cells of a myocardiac cell lineage. Cells can be cultured in growth media containing appropriate growth factors, i.e., a growth factor cocktail containing proteins which facilitate or enhance the development of cardiomyocytes.

III. Compounds of the Present Invention and Methods for Their Preparation

A. The Compounds of Formula I

In one aspect, the present invention provides compounds of Formula I having the following structure: 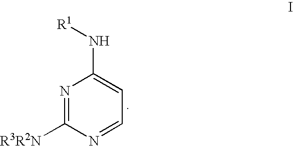
In Formula I, R¹ is a functional group including, but not limited to, hydrogen, C₁₋₄alkyl, C₃₋₈cycloalkyl, and C₀₋₂alkylaryl, substituted with 0-2 R^{1a} groups that are independently selected and are functional groups, including, but not limited to, halogen, C₁₋₄alkyl, C₁₋₄alkoxy, —OH, —N(R^{1b}, R^{1b}), —SO₂N(R^{1b}, R^{1b}), —C(O)N(R^{1b}, R^{1b}), heterocycloalkyl and —O-aryl, or when R^{1a} groups are on adjacent ring atoms, they are optionally taken together to form a functional group including, but not limited to, —O—(CH₂)₁₋₂—O—, —O—C(CH₃)₂CH₂— and —(CH₂)₃₄—, or R¹ is optionally taken together with the nitrogen to which it is attached to form a heterocycle, optionally substituted with C₁₋₄alkyl, C₃₋₈cycloalkyl, C₁₋₄alkylhydroxy and C₀₋₂alkylaryl; each R^{1b} group is independently selected and is a functional group including, but not limited to, hydrogen and C₁₋₄alkyl. In Formula I, R² is a functional group including, but not limited to, C₁₋₄alkyl, C₃₋₈cycloalkyl and C₀₋₂alkylaryl, substituted with 0-2 R^{2a} groups that are independently selected and are functional groups including, but not limited to, halogen, C₁₋₄alkyl, C₁₋₄alkoxy, —N(R^{2b}, R^{2b}), —SO₂N(R^{2b}, R^{2b}), —C(O)N(R^{2b}, R^{2b}) and —O-aryl, or when R^{2a} groups are on adjacent ring atoms, they are optionally taken together to form a functional group including, but not limited to, —O—(CH₂)₁₋₂—O—, —O—C(CH₃)₂CH₂— and —(CH₂)₃₋₄—; and each R^{2b} group is independently selected and is a functional group including, but not limited to, hydrogen and C₁₋₄alkyl. R³, in Formula I, is hydrogen, or R³ is optionally taken together with R² and the nitrogen to which both are attached to form a heterocycle, optionally substituted with, for example, C₁₋₄alkyl or C₀₋₂alkylaryl.

The compounds of the present invention include all pharmaceutically acceptable salts, isomers, solvates, hydrates and prodrugs thereof.

In one embodiment, R¹ is a functional group including, but not limited to, the following:

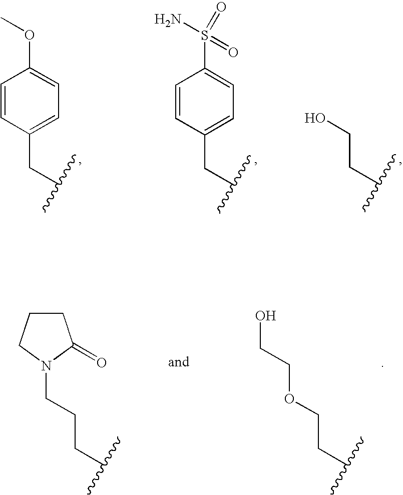

In a preferred embodiment, R¹ is

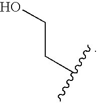

In one embodiment, R² is a functional group including, but not limited to, the following:

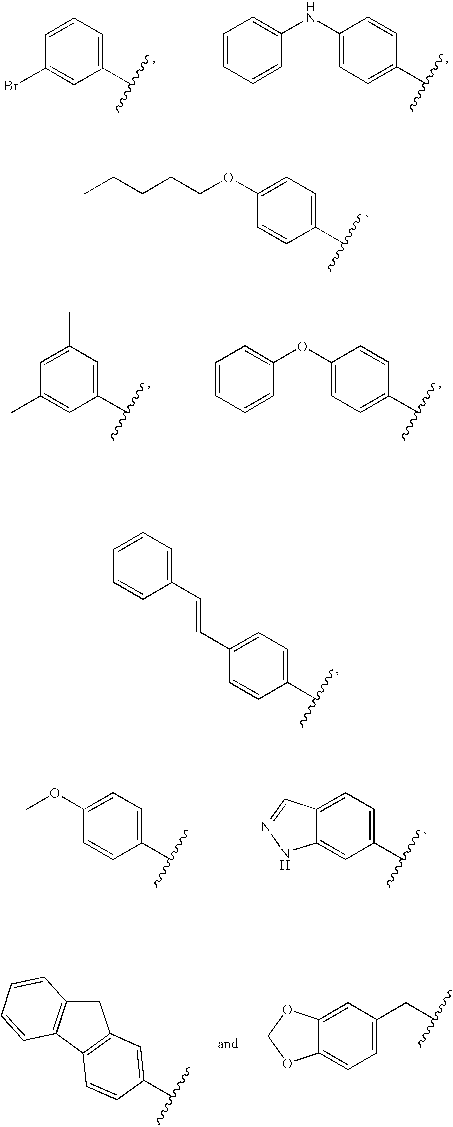

In certain preferred embodiments, R³ is hydrogen. However, in other embodiments, R² and R³ and the nitrogen to which both are attached to form a heterocycle. Examples of suitable heterocycles include, but are not limited to, the following:

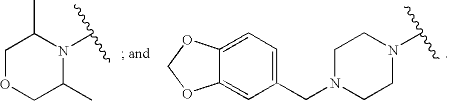

In one preferred embodiment, the compounds of the present invention have the following general structure:

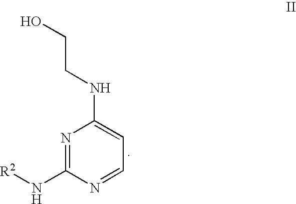

In Formula II, above, R² is as defined above with respect to Formula I. In preferred embodiments, R² of Formulae I and II include, but are not limited to, the following:

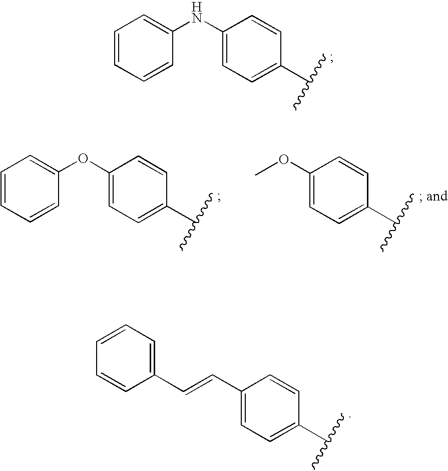

Preferred compounds of the present invention include, but are not limited to, the following (which are referred to herein as Cardiogenol A, B, C and D, respectively):

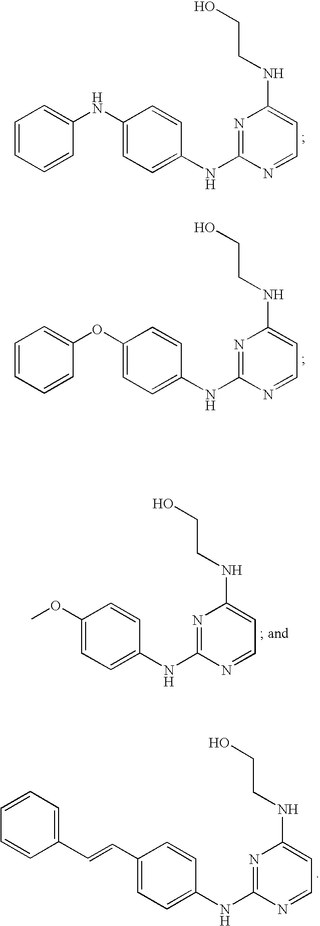

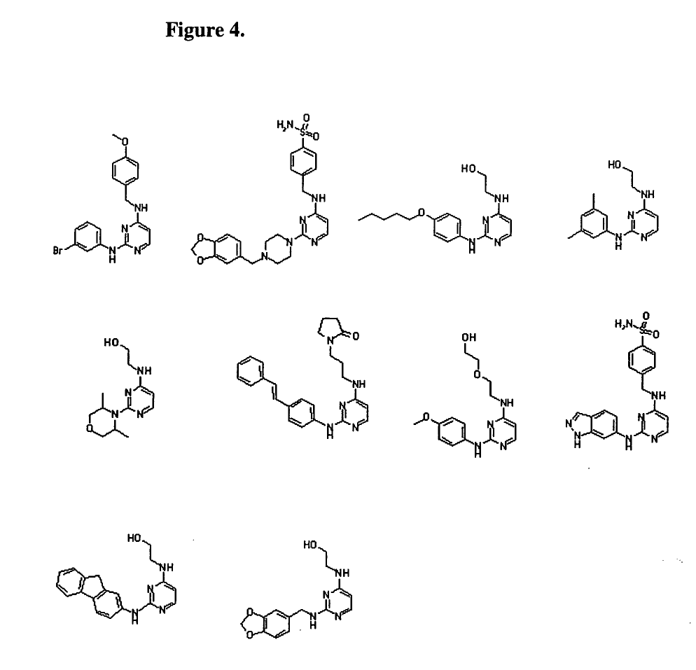

Other preferred compounds of the present invention include, but are not limited to, those exemplar compounds set forth in FIG. 4.

The compounds of Formula I and II can be readily screened for their ability to induce cardiomyogenesis using the in vitro and in vivo screening methods set forth below and, in particular, in the examples.

B. Preparation of Compounds

The compounds of the present invention can be prepared by either solid-phase or solution-phase synthesis.

1. Solid-Phase Synthesis

Methods directed to the solid-phase synthesis of the compounds of Formulae I and II are discussed herein in Example I, as well as in Ding et al., J. Am. Chem. Soc., 124(8):1594 (2002), Ding et al., J. Am. Chem. Soc., 124(49):14520-14521 (2002) and in U.S. patent application Ser. No. 10/687,220, filed on Oct. 15, 2003, U.S. Patent Application No. 60/328,763, filed Oct. 12, 2001, U.S. Patent Application No. 60/331,835, filed Nov. 20, 2001, U.S. Patent Application No. 60/346,480, filed Jan. 7, 2002, U.S. Patent Application No. 60/348,089, filed Jan. 10, 2002, and U.S. patent application Ser. No. 10/270,030, filed Oct. 12, 2002 (bearing Attorney Docket No. 21288-000340), the teachings of all of which are incorporated herein by reference for all purposes.

Generally, 2-ethanolamine is coupled to (4-formyl-3,5-dimethoxyphenoxy)methyl polystyrene resin (PAL-resin) by reductive amination. PAL-resin (1 g, 1.1 mmole) is suspended in DMF (4 mL) and ethanolamine (5.5 mmole), acetic acid (0.65 mL, 1.13 mmole) and sodium triacetoxyborohydride (720 mg, 3.4 mmole) are then added into the solution. The mixture is shaken gently at room temperature for about 12 hours. The resulting resin is then washed, for example, with DMF (10 mL, 3 times), methanol (10 mL, 3 times) and dichloromethane (10 mL, 3 times). The aniline bound resin is then reacted with 2,4-dichloropyrimidine (2.2 mmole) and diisopropylethylamine (0.5 ml, 3 mmole) in 1-butanol (5 mL) at 80° C. for 12 hours. The resulting resin is then washed as described above.

Pyrimidine bound PAL resin (100 mg, 0.1 mmole) is mixed with, for example, different aromatic amines (1.0 mmole) in 11 mL butanol. The reaction mixture is heated at 120° C. for about 12 h to yield desired products. The resulting resin is then washed as described above and cleaved with CH₂Cl₂:TFA:Me₂S:H₂O/45:45:5:5 (v/v/v/v, 0.5 mL) at room temperature for approximately 2 hours. The solution is collected and dried in vacuo to afford the desired crude product. The crude products are then easily purified using, for example, preparative RP-HPLC with H₂O (with 0.1% TFA) and MeCN as solvents.

It will be readily apparent to those of skill in the art that similar solid-phase synthesis techniques can be used to prepare the other compounds of Formulae I and II.

2. Solution-Phase Synthesis

The compounds of the present invention can be prepared by solution-phase synthesis as set forth in Example 1. Typically, the solution-phase synthesis of the compounds of Formula I involves first substituting a 2,4-dihaloheteroaryl (such as a 2,4-dichoropurine) with a suitable substituent (such as a hydroxyethylamino group) under appropriate reaction conditions known to one of skill in the art. This is followed by substitution with a second suitable substituent (such as a suitably substituted aniline (e.g., (4-phenyl amino) aniline, 4-phenoxyaniline, 4-methoxyaniline, 4-amino-trans-stilnene, etc.) under appropriate reaction conditions known to one of skill in the art. The compounds of the present invention can be purified using standard methods (such as preparative RP-HPLC) known to those of skill in the art.

IV. Use of the Compounds/Compositions to Induce Cardiomyogenesis

The compositions of the present invention can be used to induce cardiomyogenesis in mammalian cells. Generally speaking, a mammalian cell is contacted with a compound of Formula I, whereupon the mammalian cell differentiates into a cell of a myocardiac lineage. The mammalian cell can be contacted with a compound of Formula I (or a composition thereof) either in vivo or in vitro. For example, cardiogenol C could be administered directly to injured or malfunctioning cardiac muscle intravenously or by direct administration during surgery.

A. In Vivo Induction of Cardiomyogenesis

The compounds of Formula I as well as compositions thereof can conveniently be used to induce cardiomyogenesis in vivo. The compounds and compositions of the present invention are administered to an individual, e.g., a mammal such as a human, in an amount effective to induce differentiation of mammalian cells into cells of a myocardiac lineage. In view of their ability to induce cardiomyogenesis, the compounds of Formula I are useful for the repair of damaged myocardium in acute heart diseases and for treating disorders such as cardiomyopathy. In a preferred embodiment, the compounds and compositions of the present invention are used to generate cardiomyocytes for the purpose of studying the development of cardiac muscle tissue. In another preferred embodiment, the compounds and compositions of the present invention are used during the treatment of a subject in need of repair or augmentation of damaged or weakened cardiac muscle tissue. In another embodiment, the compositions of the present invention are used to treat a subject who desires augmentation or enhancement of cardiac muscle tissue that is not damaged or weakened. Such subjects can include, for example, those at risk for cardiac diseases or disorders.

One of skill in the art will appreciate that the compositions of the present invention can be used alone or in combination with other compounds and therapeutic regimens to induce cardiomyogenesis. For example, a compound of Formula I may be administered in conjunction with purified or synthesized growth factors and other agents, or combinations thereof, which enhance the development of cardiac muscle tissue.

An effective amount of the composition will be determined by the existence, nature, and extent of any adverse side-effects that accompany the administration of the composition; the LD50 of the composition; and the side-effects of the composition at various concentrations. Typically, the amount of the composition administered will range from about 0.01 to about 20 mg per kg, more typically about 0.05 to about 15 mg per kg, even more typically about 0.1 to about 10 mg per kg body weight.

The compositions can be administered, for example, by intravenous infusion, orally, intraperitoneally, or subcutaneously. Oral administration is the preferred method of administration. The formulations of compounds can be presented in unit-dose or multi-dose sealed containers, such as ampoules and vials.

The compositions of the present invention are typically formulated with a pharmaceutically acceptable carrier before administration to an individual or subject. Pharmaceutically acceptable carriers are determined, in part, by the particular composition being administered (e.g., cardiogenol C), as well as by the particular method used to administer the composition. Accordingly, there are a wide variety of suitable formulations of pharmaceutical compositions of the present invention (see, e.g., Remington's Pharmaceutical Sciences, 17th ed., 1989).

Formulations suitable for oral administration can consist of (a) liquid solutions, such as an effective amount of the compound of Formula I suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of the following: lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compositions of the present invention may be in formulations suitable for other routes of administration, such as, for example, intravenous infusion, intraperitoneally, or subcutaneously. The formulations include, for example, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can be prepared from sterile powders, granules, and tablets.

The dose administered to a patient, in the context of the present invention should be sufficient to effect a beneficial therapeutic response in the patient over time. For example, if the compositions of the present invention are administered to treat or prevent cardiomyopathy, the dose administered to the patient should be sufficient to prevent, retard, or reverse the diminished capacity of the cardiac muscle to rhythmically contract. The dose will be determined by the efficacy of the particular composition employed and the condition of the patient, as well as the body weight or surface area of the patient to be treated. The size of the dose also will be determined by the existence, nature, and extent of any adverse side-effects that accompany the administration of a particular composition in a particular patient.

B. In Vitro Induction of Cardiomyogenesis

The compositions of the present invention can conveniently be used to induce cardiomyogenesis in vitro. Mammalian cells are contacted with the compositions, whereupon the mammalian cells differentiates into cells of a myocardiac lineage.

1. Suitable Cells

The cells to be differentiated into cells of a myocardiac lineage can be derived from any suitable mammal. For example, the cells can be obtained from rodents such as, for example, mice, rats, guinea pigs, and rabbits; non-rodent mammals such as, for example, dogs, cats, pigs, sheep, horses, cows, and goats; primates such as, for example, chimpanzees and humans. The cells to be differentiated may be primary cells or may be cells maintained in culture. If the cells are maintained in culture, they are typically contacted with the compounds/compositions of the present invention between the 12th and 15th passage in culture. Techniques and methods for establishing a primary culture of cells for use in the methods of the invention are known to those of skill in the art (see, e.g., Humason, ANIMAL TISSUE TECHNIQUES, 4^{th} ed., W. H. Freeman and Company (1979), and Ricciardelli et al., In Vitro Cell Dev. Biol., 25:1016 (1989)).

Human mesenchymal stem cells (MSC) may be obtained by isolating pluripotent mesenchymal stem cells from other cells in the bone marrow or other MSC source. Bone marrow cells may be obtained from iliac crest, femora, tibiae, spine, rib or other medullary spaces. Other sources of human mesenchymal stem cells include embryonic yolk sac, placenta, umbilical cord, fetal and adolescent skin, blood, adipose tissue, and muscle satellite cells. Typically, cells from a tissue specimen containing mesenchymal stem cells are cultured in growth medium containing growth factors that (1) stimulate mesenchymal stem cell growth without differentiation, and (2) allow for the selective adherence of only the mesenchymal stem cells to a substrate surface. After culturing the cells for a suitable amount of time, non-adherent matter is removed from the substrate surface, thus providing an expanded population of mesenchymal stem cells. Thus, homogeneous MSC populations are obtained by positive selection of adherent marrow or periosteal cells which are free of markers associated with either hematopoietic cell or differentiated mesenchymal cells.

Preferably, the mammalian cells contacted by the compounds of the invention are stem cells, particularly embryonic stem cells (ESCs). Methods for isolation of human and animal ESCs are well known in the art. See, e.g., Brook F A, Proc. Natl. Acad. Sci. USA, 94:5709-12 (1997); Grounds et al., J. Histochem. and Cytochem., 50:589-610 (2002); Reubinoff, Nat. Biotech., 18:399-404 (2000). Mammalian embryonic stem cells include, for example, murine R1 cells and human embryonic stem cells.

2. General Culturing Methods

The mammalian cells (e.g., ESCs) may be contacted with a compound of Formula I alone, in combination with other compounds of Formula I, either together in a single mixture or sequentially, or in the presence of other growth factors. Those of skill in the art will appreciate that the amount of the compounds, e.g., the amount of any cardiogenol A, B, C or D, and growth factors can be adjusted to facilitate induction of differentiation in particular cell types. For example, the amount of a cardiogenol contacted with the cells is typically from about 0.01 μM (52 ng/ml) to about 10 μM (2.6 μg/ml), more typically from about 0.02 μM to about 5 μM, even more typically from about 0.05 μM to about 1 μM, yet more typically from about 0.075 μM to about 0.5 μM, and most typically at about 1 μM.

This aspect of the present invention relies upon routine techniques in the field of cell culture. Suitable cell culture methods and conditions can be determined by those of skill in the art using known methodology (see, e.g., Freshney et al., CULTURE OF ANIMAL CELLS (3rd ed. 1994)). In general, the cell culture environment includes consideration of such factors as the substrate for cell growth, cell density and cell contract, the gas phase, the medium, and temperature.

Incubation of cells is generally performed under conditions known to be optimal for cell growth. Such conditions may include, for example, a temperature of approximately 37° C. and a humidified atmosphere containing approximately 5% CO₂. The duration of the incubation can vary widely, depending on the desired results. In general, incubation is preferably continued until the cells express suitable Proliferation is conveniently determined using ³H thymidine incorporation or BrdU labeling.

Plastic dishes, flasks, or roller bottles may be used to culture cells according to the methods of the present invention. Suitable culture vessels include, for example, multi-well plates, Petri dishes, tissue culture tubes, flasks, roller bottles, and the like.

Cells are grown at optimal densities that are determined empirically based on the cell type. Cells are typically passaged 12-15 times and discarded after 15 passages.

Cultured cells are normally grown in an incubator that provides a suitable temperature, e.g., the body temperature of the animal from which is the cells were obtained, accounting for regional variations in temperature. Generally, 37° C. is the preferred temperature for cell culture. Most incubators are humidified to approximately atmospheric conditions.

Important constituents of the gas phase are oxygen and carbon dioxide. Typically, atmospheric oxygen tensions are used for cell cultures. Culture vessels are usually vented into the incubator atmosphere to allow gas exchange by using gas permeable caps or by preventing sealing of the culture vessels. Carbon dioxide plays a role in pH stabilization, along with buffer in the cell media and is typically present at a concentration of 1-10% in the incubator. The preferred CO₂ concentration typically is 5%.

Defined cell media are available as packaged, premixed powders or presterilized solutions. Examples of commonly used media include MEM-α, DME, RPMI 1640, DMEM, Iscove's complete media, or McCoy's Medium (see, e.g., GibcoBRL/Life Technologies Catalogue and Reference Guide; Sigma Catalogue). Typically, MEM-α or DMEM are used in the methods of the invention. Defined cell culture media are often supplemented with 5-20% serum, typically heat inactivated serum, e.g., human, horse, calf, and fetal bovine serum. Typically, 10% fetal bovine serum is used in the methods of the invention. The culture medium is usually buffered to maintain the cells at a pH preferably from about 7.2 to about 7.4. Other supplements to the media typically include, e.g., antibiotics, amino acids, and sugars, and growth factors.

C. Detection of Cardiomyogenesis

After administration of the compositions of the present invention in vivo or in vitro, the induction of cardiomyogenesis can be detected by a number of different methods including, but not limited to: detecting expression of cardiomyocyte-specific proteins, detecting expression of cardiac muscle cell-specific transcription factors, detecting expression of proteins essential for cardiac muscle function, and detecting the beating of cardiac muscle cells. Specific examples of cardiomyocyte-specific proteins and cardiac muscle cell-specific transcription factors are described herein.

1. Detection of Cardiomyocyte-Specific Proteins

Expression of cardiac muscle cell differentiation can be detected by measuring the level of a cardiac muscle cell-specific protein or mRNA. The level of particular cardiac muscle cell-specific proteins can conveniently be measured using immunoassays, such as immunohistochemical staining, western blotting, ELISA and the like with an antibody that selectively binds to the particular cardiomyocyte-specific proteins or a fragment thereof. Detection of the protein using protein-specific antibodies in immunoassays is known to those of skill in the art (see, e.g., Harlow & Lane, Antibodies: A Laboratory Manual (1988), Coligan, Current Protocols in Immunology (1991); Goding, Monoclonal Antibodies: Principles and Practice (2d ed. 1986); and Kohler & Milstein, Nature 256:495-497 (1975). For measurement of mRNA, amplification, e.g., PCR, LCR, or hybridization assays, e.g., northern hybridization, RNAse protection, dot blotting, are preferred. The level of protein or mRNA is detected, for example, using directly or indirectly labeled detection agents, e.g., fluorescently or radioactively labeled nucleic acids, radioactively or enzymatically labeled antibodies. These assays are well-known to those of skill in the art and described in, e.g., Ausubel, et al. ed. CURRENT PROTOCOLS IN MOLECULAR BIOLOGY (2001).

Typically, expression of the cardiac muscle cell protein ANF is used to detect differentiated cardiomyocytes. ANF is a polypeptide hormone that is synthesized primarily in cardiac myocytes and is a down-stream target of several cardiomyogenesis transcriptional factors; it is considered a specific cardiomyocyte “marker” gene (Boer, Exp. Cell Res., 207:421-29 (1993). Activation of the ANF gene can be measured, for example, by inserting the ANF promoter region into a reporter plasmid upstream of a readily detectable protein or an enzyme whose activity is readily detectable, such as luciferase. An increase in the level of expression of the reporter gene in these circumstances is indicative of cardiomyocyte differentiation.

a) Immunohistochemical Detection

For direct immunohistochemical staining of cells to detect, e.g., cardiomyocyte-specific genes, cells are seeded in 96-well assay plates at a suitable density and treated with an appropriate amount of a compound of Formula I (e.g., cardiogenol A), either alone or with other growth factors for an appropriate time. Cells are then fixed in a 10% formalin solution. The fixed cells are washed again and stained with a reagent specific for the protein of interest (e.g., an antibody specific for the protein or, if an enzymatic reporter gene is used, a reagent whose detectability by, e.g., fluorometric methods changes in the presence of the reporter gene enzyme) using methods known to those of skill in the art (see, e.g., Harlow & Lane, 1988, supra; Coligan, 1991, supra; Goding, 1986, supra; and Kohler & Milstein, 1975, supra). Photographic images of the cells are taken and positive cells expressing the cardiomyocyte-specific gene are counted manually from the images.

2. Detection of Cardiac Muscle Cell-Specific Transcription Factors

Expression of cardiac muscle cell-specific transcription factors can be detected using reporter gene assays. A variety of reporter gene assays are well known to those of skill in the art. See, e.g., New et al., Phytother. Res., 17:439-48 (2003); Schenborn et al, Mol. Biotechnol., 13:2944 (1999). Reporter genes such as, for example, chloramphenicol acetyltransferase, firefly luciferase, bacterial luciferase, or β-galactosidase can be used in the reporter gene assays. The reporter construct is typically transiently or stably transfected into a cell. The promoter region of the relevant gene is typically amplified by PCR appropriate primers. The resulting PCR product is inserted into a suitable cloning vector, amplified and sequenced. The resulting plasmid is digested with appropriate restriction enzymes and the resulting fragment is inserted into a vector comprising a reporter gene.

a) Transiently Transfected Cells

For reporter gene assays with transiently transfected cells, the cells are typically seeded in a 6-well plate at a density of approximately 30,000 cells/well in 2 mL of growth medium an incubated overnight or for a suitable time. Plasmid DNA is transfected into the cells using a suitable transfection reagent. After 8 hours, the transfected cells are plated into 96-well assay plates (e.g., Corning) and treated with an appropriate amount of a compound of Formula I (e.g., cardiogenol A). The cells are incubated for 4 days, then the reporter gene activity in the cells is assayed using methods known to those of skill in the art.

b) Stably Transfected Cells

For reporter gene assays with stably transfected cells, the cells are typically seeded in a 6-well plate at a density of approximately 30,000 cells/well in 2 mL of growth medium an incubated overnight or for a suitable time. An appropriate amount of reporter plasmid and a vector comprising a selectable marker (e.g., an antibiotic resistance gene) are co-transfected into the cells using an appropriate transfection reagent. After an appropriate incubation time, cells are seeded in a 10 cm culture dish and an appropriate amount of antibiotic is added to the culture medium. Fresh antibiotic is added at appropriate intervals. The antibiotic resistant colonies are pooled to yield the stably transfected cells. The transfected cells are plated into 96-well assay plates (e.g., Corning) and treated with an appropriate amount of a compound of Formula I (e.g., cardiogenol A). The cells are incubated for 4 days, then the reporter gene activity in the cells is assayed using methods known to those of skill in the art.

3. Administration of Differentiated Cardiomyocytes

Differentiated cardiomyocytes can be administered to a subject by any means known to those of skill in the art. In one embodiment of the invention, differentiated cardiomyocytes on an intact solid support (e.g., a three-dimensional matrix or a planar surface) can be administered to the subject, e.g., via surgical implantation. Alternatively, the differentiated cardiomyocytes can be detached from the matrix, i.e., by treatment with a protease, before administration to the subject, e.g., intravenous, subcutaneous, or intraperitoneal.

In some embodiments of the present invention, embryonic stem cells are extracted and subsequently contacted with a matrix for proliferation and differentiation into cells of a myocardiac lineage. Cells can be extracted from the subject to be treated, i.e., autologous (thereby avoiding immune-based rejection of the implant), or can be from a second subject, i.e., heterologous. In either case, administration of cells can be combined with an appropriate immunosuppressive treatment.

Cardiomyocytes differentiated according to the methods of the present invention may be administered to a subject by any means known in the art. Suitable means of administration include, for example, intravenous, subcutaneous, intraperitoneal, and surgical implantation. The cardiomyocytes may be directly injected into cardiac muscle or applied topically, for example, during surgery on the heart.

The cells may be in formulations suitable for administration, such as, for example, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can be prepared from sterile powders, granules, and tablets.

For surgical implantation, differentiated cells are typically left on an intact solid support, e.g., a three-dimensional matrix or planar surface. The matrix or planar surface is surgically implanted into the appropriate site in a subject. For example, a patient needing a replacement of a portion of cardiac muscle tissue can have differentiated cells on an intact solid support surgically implanted.

In determining the effective amount of the cells to be administered in the treatment or prophylaxis of conditions owing to diminished or malfunctioning cardiac muscle cells, the physician evaluates cell toxicity, transplantation reactions, progression of the disease, and the production of anti-cell antibodies. For administration, cardiomyocytes differentiated according to the methods of the present invention can be administered in an amount effective to provide cardiac muscle cells to the subject, taking into account the side-effects of the cardiomyocytes at various concentrations, as applied to the mass and overall health of the patient. Administration can be accomplished via single or divided doses.

### EXAMPLES

The following examples are offered to illustrate, but not to limit, the claimed invention.

### Example 1

### Synthesis and Characterization of Cardiogenol A, B, C, and D

All chemicals used for synthesis were purchased from Aldrich. 2,4-Dichloropyrimidine (200 mg, 1.34 mmole) was dissolved in 5 mL of ethanol and 282 μL (1.62 mmole) of diisopropylethylamine (DIEA) and 90 mg (1.47 mmole) of ethanolamine were added into the solution. The reaction mixture was then heated at 50° C. for 12 h to afford 2-chloro-4-(1-hydroxylethylamino)-pyrimidine (80% yield). 20 mg (0.12 mmole) of 2-chloro-4-(1-hydroxylethylamino)-pyrimidine was dissolved in 1-butanol (1 mL) and 42.4 mg (0.23 mmole) of 4-(phenylamino) aniline was added. The reaction mixture was heated at 200° C. for 15 min in microwave reactor to afford cardiogenol A (85% yield). Similarly, use of 42.7 mg (0.23 mmole) of 4-phenoxyaniline, 28.4 mg (0.23 mmole) of 4-methoxyaniline or 45.0 mg (0.23 mmole) of 4-amino-trans-stilbene will afford cardiogenol B (80% yield), C (90% yield) or D (75% yield), respectively. The compounds were purified by preparative HPLC using H₂O (with 0.1% TFA) and MeCN as solvents with a linear gradient of 5% to 90% MeCN in 10 min. The desired peaks were collected and freeze-dried to give final products.

Cardiogenol A: ¹H NMR (400 MHz, DMSO): δ (ppm) 3.44 (m, 2H), 3.57 (m, 2H), 6.20 (d, 1H, J=7.2), 6.83 (t, 1H, J=7.2), 7.08 (m, 5H), 7.23 (t, 2H, J=8.3), 7.34 (d, 2H, J=7.7), 7.67 (d, 1H, J=6.6), 8.25 (s, 1H), 8.95 (s, 1H), 10.18 (s, 1H). High Resolution Mass Spectrometry (MALDI-FTMS): Calculated [MH⁺] (C₁₈H₂₀N₅O) 322.1662, found 322.1660.

Cardiogenol B: ¹H NMR (400 MHz, DMSO): δ (ppm) 3.45 (m, 2H), 3.56 (m, 2H), 6.24 (d, 1H, J=7.2), 7.03 (m, 5H), 7.14 (t, 1H, J=7.4), 7.40 (t, 2H, J=7.5), 7.55 (d, 2H, J=8.6), 7.73 (d, 1H, J=7.0), 8.97 (s, 1H), 10.30 (s, 1H). High Resolution Mass Spectrometry (MALDI-FTMS): Calculated [MH⁺] (C₁₈H₁₉N₄O₂) 323.1502, found 323.1498.

Cardiogenol C: ¹H NMR (400 MHz, DMSO): δ (ppm) 3.42 (m, 2H), 3.55(m, 2H), 3.72 (s, 3H), 6.21 (d, 1H, J=7.2), 6.97 (d, 2H, J=8.9), 7.41 (d, 2H, J=8.4), 7.66 (d, 1H, J=7.1), 8.93 (s, 1H), 10.07 (s, 1H). High Resolution Mass Spectrometry (MALDI-FRMS): Calculated [MH⁺] (C₁₃H₁₇N₄O₂) 261.1346, found 261.1342.

Cardiogenol D: ¹H NMR (400 MHz, DMSO): δ (ppm) 3.48 (m, 2H), 3.62 (m, 2H), 6.26 (d, 1H, J=7.2), 7.27 (m, 3H), 7.38 (t, 2H, J=7.5), 7.61 (m, 6H), 7.77 (d, 1H, J=7.0), 9.00 (s, 1H), 10.35 (s, 1H). High Resolution Mass Spectrometry (MALDI-FTMS): Calculated [MH⁺] (C₂₀H₂₁N₄O) 333.1710, found 333.1711.

In addition to the foregoing solution-phase synthesis methods, the compounds of the present invention can also be made using solid-phase synthesis methods as follows:

Generally, 2-ethanolamine was coupled to (4-formyl-3,5-dimethoxyphenoxy)methyl polystyrene resin (PAL-resin) by reductive amination. PAL-resin (1 g, 1.1 mmole) was suspended in DMF (4 mL) and ethanolamine (5.5 mmole), acetic acid (0.65 mL, 1.13 mmole) and sodium triacetoxyborohydride (720 mg, 3.4 mmole) were then added into the solution. The mixture was shaken gently at room temperature for 12 hours. The resulting resin was then washed with DMF (10 mL, 3 times), methanol (10 mL, 3 times) and dichloromethane (10 mL, 3 times). The aniline bound resin was then reacted with 2,4-dichloropyrimidine (2.2 mmole) and diisopropylethylamine (0.5 mL, 3 mmole) in 1-butanol (5 mL) at 80° C. for 12 hours. The resulting resin was then washed as described above.

Pyrimidine bound PAL resin (100 mg, 0.1 mmole) was mixed with different aromatic amines (1.0 mmole) in 11 mL butanol. The reaction mixture is heated at 120° C. for 12 h to yield desired products. The resulting resin was then washed as described above and cleaved with CH₂Cl₂:TFA:Me₂S:H₂O/45:45:5:5 (v/v/v/v, 0.5 mL) at room temperature for 2 hours. The solution was collected and dried in vacuo to afford the desired crude product. The crude products can then be purified using preparative RP-HPLC using H₂O (with 0.1% TFA) and MeCN as solvents.

### Example 2

### Cell Culture and High Throughput Screen for Cardiomyogenesis Inducing Molecules

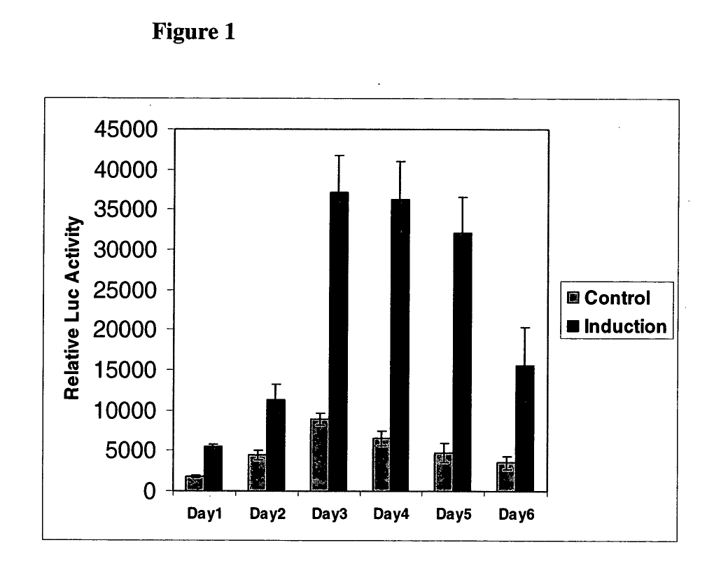

P19 embryonic carcinoma (EC) cells (from ATCC) were cultured in MEM-alpha with 7.5% new born calf serum and 2.5% FBS (Gibco) at 37° C. in 5% CO₂. P19CL6 cells (a gift from Dr. Michael Schneider and Dr. Nakamura Teruya) were cultured in MEM-alpha with 10% FBS (from Gibco) at 37° C. in 5% CO₂. A fragment (˜700 bps) containing rat ANF promoter region was amplified by using PCR primers (5′-ccgacgcgtgaaacatcacattggttgcctt and 5′-ccgctcgagcactctctggtttctctctc) and then subcloned into the PGL3-BV luciferase reporter plasmid using MluI and XhoI restriction sites. A stable P19 clone harboring the reporter plasmid afforded a 5- to 7-fold increase (FIG. 1) in luciferase signal upon standard cardiomyogenesis differentiation conditions for P19 cells (EB formation and treatment with 1% DMSO (see Skerjank I S, Trends Cardiovasc Med, 9:139-143 (1999)). This cell line was used to screen a 100,000 compound heterocycle library in a monolayer format according to the following method. 103 Cells were plated in each well of 384-well plates with 100 μL induction medium (MEM-alpha with 5% FBS); 500 nL of 1 mM compound solution was then added into each well. After compound treatment for 3 days, the medium was changed with no additional compounds added. The luciferase activity was measured after 7 days of compound treatment using the Bright-Glo luciferase assay kit (Promega). Approximately 80 compounds were identified that up-regulated luciferase activity >4-fold in the absence of EBs.

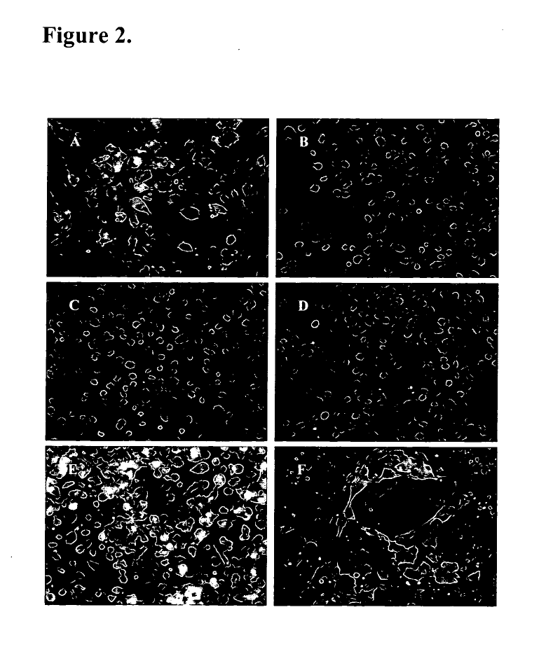

MHC is one of the essential motor proteins responsible for cardiac muscle contractibility and was used as a secondary assay for differentiation. Thirty five of the 80 compounds identified in the screening assay described above also induced sarcomeric myosin heavy chain (MHC) expression in P19CL6 cells. The P19CL6 cell line is a subclone of P19 EC cells with higher potential for cardiomyogenesis. See Habara-Ohkubo, Cell Struct. Funct., 21:101-110 (1996). MHC expression was determined in P19CL6 cells by immunostaining cells with anti-MHC antibody (MF20) (FIG. 2F).

### Example 3

### Identification of Cardiogenols A, B, C and D as Compounds with Cardiomyogenesis Inducing Activity

Among the thirty five compounds identified in the previous example, four diaminopyrimidines, cardiogenol A-D (Table 1), were the most potent in inducing MHC expression.

TABLE 1

| | R² | | | |
|---|---|---|---|---|
| Compound Name | 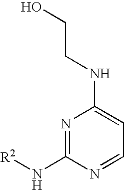 | EC₅₀ | Optimal Activity | Toxicity EC₅₀ |
| Cardiogenol A | 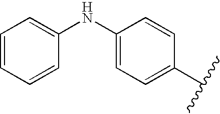 | 1 μM | ++ | 5 μM |
| Cardiogenol B | 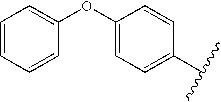 | 0.5 μM | +++ | 5 μM |
| Cardiogenol C | 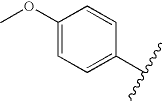 | 0.1 μM | ++++ | 25 μM |
| Cardiogenol D | 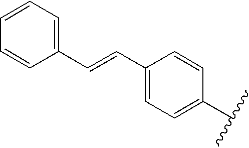 | 0.1 μM | ++++ | 2.5 μM |

The optimal activities of the cardiogenols in Table 1 are indicated by a series of "+" signs, as follows: ++: 10-25% cells are positive for MHC after 7 days; +++: 25-40% cells are positive for MHC after 7 days; ++++: 40-55% cells are positive for MHC after 7 days.

To confirm that these compounds are general cardiomyogenesis inducing agents, their effects on undifferentiated R1 mouse ESCs were analyzed. R1 mouse ESCs can be maintained in a pluripotent state with the addition of leukemia inhibitory factor (LIF) in the culture medium. The embryonic stem cell line R1 was cultured in gelatin-coated tissue culture dishes with Knockout DMEM with 15% ES serum replacement, 1 mM L-glutamine (from Gibco), 1% nonessential amino acids stock, 1% nucleosides stock, 0.1 mM beta-mercaptomethanol (from Specialty Media) and 1000 units/mL of leukemia inhibitory factor (LIF, from Chemicon).

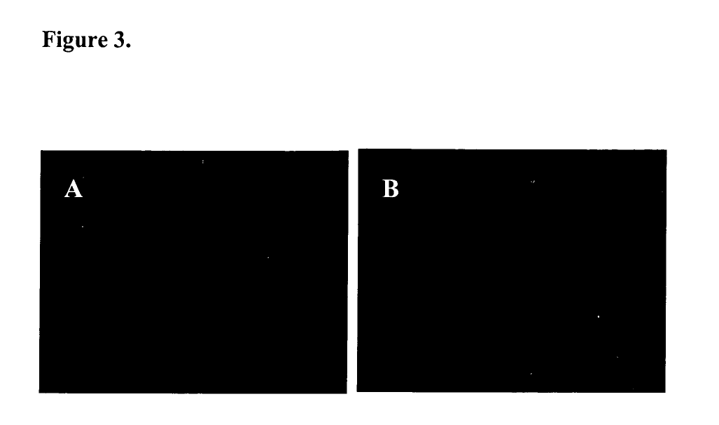

For differentiation, R1 cells were plated in a monolayer (10000 cells/well) in gelatin-coated 384-well or 96-well plates with 100 μL of DMEM with 10% FBS and 0.25 μM of compounds. LIF was not present during differentiation. After 7 days in culture (3 days with compounds and then, after changing the medium without additional compound added, another 4 days), the presence of beating cardiac muscle was visualized under a microscope. In addition to the expression of MHC (FIG. 2A), the cardiac specific gene, GATA-4, was detected by immunofluorescent staining using anti-GATA4 antibody (FIG. 2B). GATA-4 is a transcription factor restricted to developing heart and it's over expression enhances cardiomyogenesis in P19 cells (Grepin et al., Development, 124:2387-95 (1997); Chadron et al., Cell and Dev. Biol., 10:85-91 (1999); Gag et al., Nature, 424:443-447 (2003). As shown in FIG. 3, neither MHC nor GATA-4 is expressed in undifferentiated R1 mouse ESCs. It was also observed that compound treatment slowed cellular proliferation with no significant cell death, indicating that this process is not simply a selection for cardiac precursor cells with the death of cells in other lineages.

### Example 4

### Cardiogenol C is a Potent Inducer of Cardiomyogenesis in Embryonic Stem Cells

Cardiogenol C has a p-metonym aniline substituting at the pyrimidine C2 position and is very potent with an EC₅₀ of 0.1 μM for inducing the differentiation of MHC positive cardiomyocytes from ESCs. Cardiogenol C showed significant cellular toxicity only at concentrations greater than 25 μM, after treating R1 cells 0.25 μM compound for 3 days and further culturing in medium without compound for 4 days, more than 50% cells stained positive for MHC and more than 90% cells are positive for GATA-4, consistent with the previous observation that GATA-4 is expressed earlier than MHC. See Boheler et al., Circ. Res., 91:189-201 (2002). Moreover, there were many beating areas in R1 cells treated with cardiogenol C, demonstrating that these MHC positive cells can form functional cardiac muscle. These results indicate that majority of the cell population was induced by cardiogenol C to differentiate into cardiac lineage (in the absence of aggregation and EB formation). This is in contrast to the current standard method of inducing cardiomyogenesis of ESCs by aggregation and formation of EBs, which results in only 5% of the cell population forming cardiomyocytes. See Boheler et al., (2002).

### Example 5

### Detection of Cardiac Muscle Cell-Specific Transcription Factors in ESCs Differentiated Using Cardiogenol C

To further characterize the activity of Cardiogenol C, the expression of the cardiac muscle cell specific transcription factors MEF2 and Nkx2.5 was examined (FIGS. 2C and D). Members of the MEF2 family are essential for muscle development. See, e.g., Edmondson, et al., Development, 1251-1263 (1994); Lin et al., Science, 276:1404-1407 (1997). Nkx2.5 together with GATA-4 regulates the expression of multiple cardiac muscle specific genes (e.g., myosin light chain 2V, atria natriuretic factor, eHAND and homeodomain transcription factor HOP). See, e.g., Small et al., Cell, 110:725-735 (2002); Shin et al., Cell, 110:725-35 (2002). Moreover, the targeted interruption of Nkx2.5 gene is embryonic lethal and results in arrest of cardiac development. See, e.g., Lyons et al., Genes Dev., 9:1654-66 (1995). Approximately 90% of cardiogenol C treated cells stain positive for MEF2 and Nkx2.5, further confirming that ESCs are differentiated into cardiac muscle by cardiogenol C.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A compound of Formula I having the following structure: 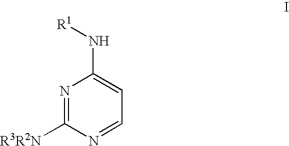
wherein:
R¹ is a member selected from the group consisting of hydrogen, C₁₋₄alkyl, C₃₋₈cycloalkyl, and CO₂alkylaryl, substituted with 0-2 R^{1a} groups that are independently selected from the group consisting of halogen, C₁₋₄alkyl, C₁₋₄alkoxy, —OH, —N(R^{1b}, R^{1b}), —SO₂N(R^{1b}, R^{1b}), —C(O)N(R^{1b}, R^{1b}), heterocycloalkyl and —O-aryl, or when said R^{1a} groups are on adjacent ring atoms they are optionally taken together to form a member selected from the group consisting of —O—(CH₂)₁₋₂—O—, —O—C(CH₃)₂CH₂— and —(CH₂)₃₄—, or R¹ is optionally taken together with the nitrogen to which it is attached to form a heterocycle, optionally substituted with C₁₋₄alkyl, C₃₋₈cycloalkyl, C₁₋₄alkylhydroxy and CO₂alkylaryl;
each R^{1b} group is a member that is independently selected from the group consisting of hydrogen and C₁₋₄alkyl;
R² is a member selected from the group consisting of C₁₋₄alkyl, C₃₋₈cycloalkyl and C₀₋₂alkylaryl, substituted with 0-2 R^{2a} groups that are independently selected from the group consisting of halogen, C₁₋₄alkyl, C₁₋₄alkoxy, —N(R^{2b}, R^{2b}), —SO₂N(R^{2b}, R^{2b}), —C(O)N(R^{2b}, R^{2b}) and —O-aryl, or when said R^{2a} groups are on adjacent ring atoms they are optionally taken together to form a member selected from the group consisting of —O—(CH₂)₁₋₂—O—, —O—C(CH₃)₂CH₂— and —(CH₂)₃₋₄—; and
each R^{2b} group is a member that is independently selected from the group consisting of hydrogen and C₁₋₄alkyl; and
R³ is hydrogen, or R² is optionally taken together with R³ and the nitrogen to which both are attached to form a heterocycle, optionally substituted with C₁₋₄alkyl or C₀₋₂alkylaryl.

2. The compound in accordance with claim 1, wherein R¹ is a member selected from the group consisting of:

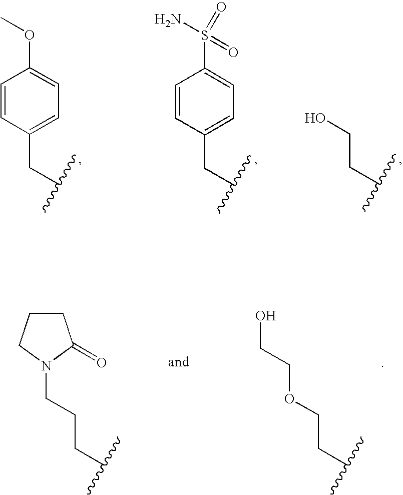

3. The compound in accordance with claim 2, wherein R¹ is

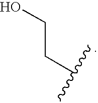

4. The compound in accordance with claim 1, wherein R² is a member selected from the group consisting of:

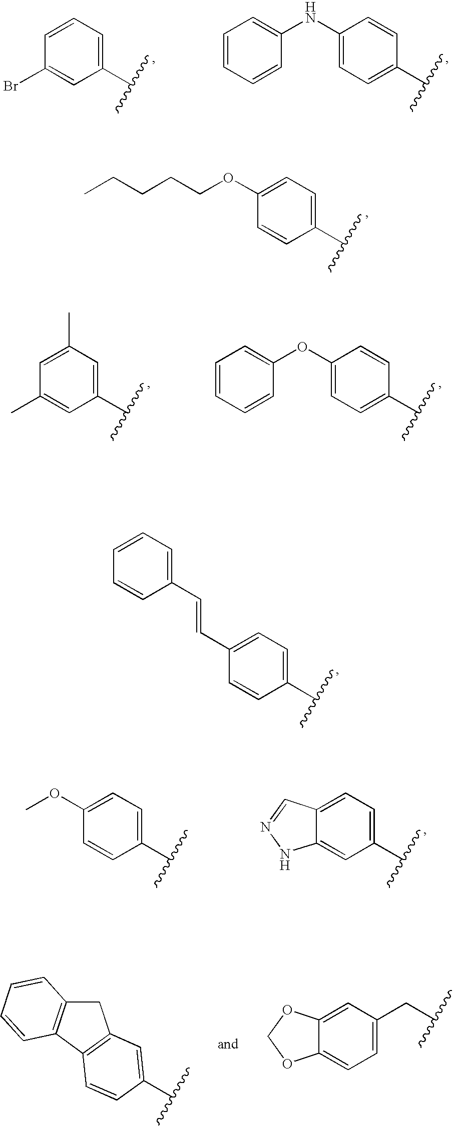

5. The compound in accordance with claim 1, wherein R³ is hydrogen.

6. The compound in accordance with claim 1, wherein R² and R³ and the nitrogen to which both are attached to form a heterocycle.

7. The compound in accordance with claim 6, wherein said heterocycle is a member selected from the group consisting of:

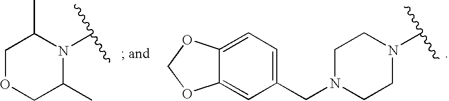

8. The compound in accordance with claim 1, wherein said compound has the following structure:

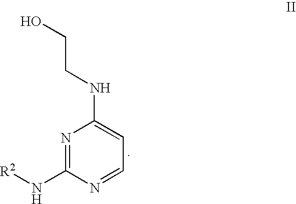

9. The compound in accordance with claim 1 or claim 8, wherein R² is a member selected from the group consisting of:

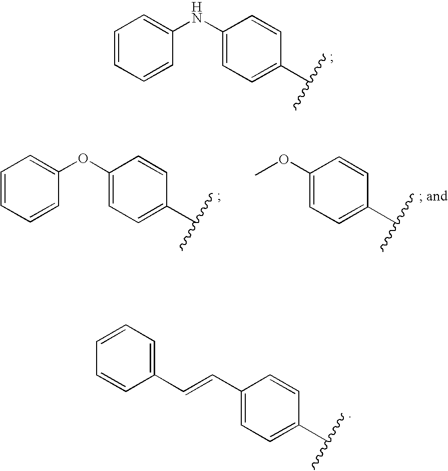

10. The compound in accordance with claim 1, wherein said compound is a member selected from the group consisting of:

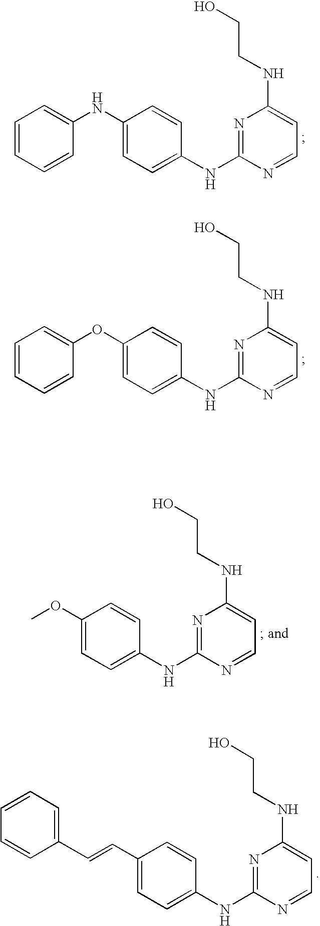

11. A pharmaceutical composition comprising a compound of claim 1 or claim 10 and a pharmaceutically acceptable carrier.

12. A method of inducing myocardiogenesis, the method comprising:
contacting a mammalian cell with a compound of claim 1, whereby the mammalian cell differentiates into a cell of myocardiac lineage.

13. The method of claim 12, wherein said compound of claim 1 is in a pharmaceutically acceptable carrier.

14. The method of claim 12, wherein the mammalian cell is in a mammal.

15. The method of claim 14, wherein the step of contacting is by oral administration of the compound to the mammal.

16. The method of claim 14, wherein the step of contacting is by intravenous administration of the compound to the mammal.

17. The method of claim 14, wherein the step of contacting is by subcutaneous administration of the compound to the mammal.

18. The method of claim 14, wherein the step of contacting is by intraperitoneal administration of the compound to the mammal.

19. The method of claim 12, further comprising detecting differentiation of the mammalian cell into a cell of a myocardiac lineage.

20. The method of claim 19, whereby differentiation of the mammalian cell into a cell of a myocardiac lineage is detected by detecting expression of a cardiomyocyte marker gene.

21. The method of claim 20, wherein the cardiomyocyte marker gene encodes atrial natriuretic factor.

22. The method of claim 19, whereby differentiation of the mammalian cell into a cell of a myocardiac lineage is detected by detecting expression of a cardiac muscle cell-specific transcription factor.

23. The method of claim 22, wherein the cardiac muscle specific transcription factor is selected from the group consisting of MEF2 and Nkx2.5.

24. The method of claim 19, whereby differentiation of the mammalian cell into a cell of a myocardiac lineage is detected by detecting expression of a motor protein involved in cardiac muscle contraction.

25. The method of claim 24, wherein the motor protein is sarcomeric myosin heavy chain motor protein.

26. The method of claim 19, whereby differentiation of the mammalian cell into a cell of a myocardiac lineage is detected by detecting expression of a cardiac specific gene.

27. The method of claim 26, wherein the cardiac specific gene is GATA-4.

28. The method of claim 12, wherein the mammalian cell is an embryonic stem cell.

29. The method of claim 28, wherein the embryonic stem cell is isolated from a mouse.

30. The method of claim 29, wherein the embryonic stem cell is an R1 embryonic stem cell.

31. The method of claim 12 wherein the mammalian cell is an embryonic carcinoma cell.

32. The method of claim 31 wherein the carcinoma cell is isolated from a mouse.

33. The method of claim 32, wherein the mouse carcinoma cell is a P19 embryonic carcinoma cell.

34. The method of claim 12, wherein the mammalian cell is a primate embryonic stem cell.

35. The method of claim 12, wherein the mammalian cell is a human embryonic stem cell.

36. The method of claim 12, wherein the mammalian cell is further contacted with a cardiomyogenesis enhancing protein.

37. The method of claim 36, wherein the cardiomyogenesis enhancing protein is a growth factor involved in cardiomyogenesis.

38. The method of claim 12, wherein the mammalian cell is attached to a solid support.

39. The method of claim 38, wherein the solid support is a three dimensional matrix.

40. The method of claim 38, wherein the solid support is a planar surface.

41. A method of inducing cardiomyogenesis, the method comprising:
contacting a mammalian cell with a compound of claim 1, whereby the mammalian cell differentiates into a cell of a myocardiac lineage.

42. The method of claim 41, wherein the mammalian cell is in a mammal.

43. The method of claim 41, wherein the step of contacting is by oral administration of the compound to the mammal.

44. The method of claim 41, wherein the step of contacting is by intravenous administration of the compound to the mammal.

45. The method of claim 41, wherein the step of contacting is by subcutaneous administration of the compound to the mammal.

46. The method of claim 41, wherein the step of contacting is by intraperitoneal administration of the compound to the mammal.

47. A method of treating a cardiac muscle disorder, the method comprising:
contacting a mammalian cell with a compound of claim 1, whereby the mammalian cell differentiates into a cell of a myocardiac lineage.

48. The method of claim 47, wherein the cardiac muscle disorder is associated with damaged myocardium.

49. The method of claim 48, wherein the cardiac muscle disorder is cardiomyopathy.

50. The method of claim 47, further comprising administering the cell of a myocardiac lineage to an individual with the disorder, thereby treating the disorder.

51. The method of claim 50, wherein the administration is by surgical implantation.
